(19) United States
(12) Patent Application Publication
(10) Pub. No.: US 2007/0141125 A1
(43) Pub. Date: Jun. 21, 2007

(54) Thermal image with antimicrobial property

(76) Inventors: Robert P. Bourdelais, Pittsford, NY (US); Cheryl J. Brickey, Greer, SC (US); David L. Patton, Webster, NY (US)

Correspondence Address: Paul A. Leipold;Patent Legal Staff, Eastman Kodak Company, 343 State Street, Rochester, NY, 14650-2201, US

(21) Appl. No.: 11/305,591

(22) Filed: Dec. 16, 2005

(51) Int. Cl. A61K 9/70; C09D 5/16

(52) U.S. Cl. 424/443; 523/122

(57) ABSTRACT

The invention relates to a packaging material comprising a substrate, an image formed by thermal dye transfer on said substrate and a transparent polymer overlayer on the opposite side of the image from said substrate, and further comprising antimicrobial composition in said overlayer.

### FIELD OF THE INVENTION

The present invention relates to an antimicrobial composition having a controlled release of an antimicrobial compound; it further relates to a pressure sensitive thermal dye transfer printed label comprising an antimicrobial composition.

### BACKGROUND OF THE INVENTION

In recent years people have become very concerned about exposure to the hazards of microbe contamination. For example, exposure to certain strains of Eschericia coli through the ingestion of undercooked beef can have fatal consequences. Exposure to Salmonella enteritidis through contact with unwashed poultry can cause severe nausea. Mold and yeast (Candida albicans) may cause skin infections. In some instances, biocontamination alters the taste of the food or drink or makes the food unappetizing. With the increased concern by consumers, manufacturers have started to produce products having antimicrobial properties. A wide variety of antimicrobial materials have been developed, which are able to slow or even stop microbial growth; such materials when applied to consumer items may decrease the risk of bacterial infection.

Noble metal ions such as silver and gold ions are known for their antimicrobial properties and have been used in medical care for many years to prevent and treat infection. In recent years, this technology has been applied to consumer products to prevent the transmission of infectious disease and to kill harmful bacteria such as Staphylococcus aureus and Salmonella. In common practice, noble metals, metal ions, metal salts, or compounds containing metal ions having antimicrobial properties may be applied to surfaces to impart an antimicrobial property to the surface. If, or when, the surface is inoculated with harmful microbes, the antimicrobial metal ions or metal complexes, if present in effective concentrations, will slow or even prevent altogether the growth of those microbes. Antimicrobial activity is not limited to noble metals but is also observed in organic materials such as triclosan, and some polymeric materials.

It is important that the antimicrobial active element, molecule, or compound be present on the surface of the article at a concentration sufficient to inhibit microbial growth. This concentration, for a particular antimicrobial agent and bacterium, is often referred to as the minimum inhibitory concentration (or MIC). It is also important that the antimicrobial agent be present on the surface of said article at a concentration significantly below that which may be harmful to the user of said article. This prevents harmful side effects of the article and decreases the risk to the user, while providing the benefit of reducing microbial contamination. More recently, metal ion exchange materials have been developed which are able to effect the so-called “controlled release” of an antimicrobial ion, by virtue of exchange of the antimicrobial ion with ions commonly present in biological environments. This approach is very general since innocuous ions such as sodium and potassium are present in virtually all biological environments. The approach has the advantage in that the antimicrobial ions are bound tightly by the ion exchange medium, but are released when exposed to conditions under which biological growth may occur.

U.S. Patent Application 20030091767 A1 to Podhajny describes a method of applying an antimicrobial treatment to a packaging material, and to polymer dispersions containing antimicrobial zeolites. The zeolite containing dispersions may be formulated in water-based or solvent-based systems. Suitable polymers for practice of the invention listed are polyamides, acrylics, polyvinyl chloride, polymethyl methacrylates, polyurethane, ethyl cellulose, and nitro celluloses.

U.S. Pat. No. 5,556,699 to Niira et al describes transparent polymeric films containing antimicrobial zeolites which are ion exchanged with silver and other ions. The films are said to display antimicrobial properties. Polymeric materials suitable for the invention include ethylene ethyl acrylate (EEA), ethylene vinyl acetate (EVA), polyethylene, polyvinyl chlorides, polyvinyl fluoride resins, and others.

There is a problem in that the polymeric binder or polymeric medium may severely limit the release of the antimicrobial material. Therefore, the exchange of antimicrobial ions from the antimicrobial films may not be facile enough to achieve a concentration of antimicrobial metal ions sufficient to limit the growth rate of a particular microbe, or may not be above the minimum inhibitory concentration (MIC). Alternatively, there is a problem in that the rate of release of antimicrobial ions from antimicrobial films may be too facile, such that the antimicrobial film may quickly be depleted of antimicrobial active materials and become inert or non-functional. Depletion results from rapid diffusion of the active materials into the biological environment with which they are in contact. It is desirable that the rate of release of the antimicrobial ions or molecules be controlled such that the concentration of antimicrobials remains above the MIC. The concentration should remain there over the duration of use of the antimicrobial article. The desired rate of exchange of the antimicrobial may depend upon a number of factors including the identity of the antimicrobial metal ion, the specific microbe to be targeted, and the intended use and duration of use of the antimicrobial article.

In recent years, thermal transfer systems have been developed to obtain prints from pictures, which have been generated electronically from a color video camera. According to one way of obtaining such prints, an electronic picture is first subjected to color separation by color filters. The respective color-separated images are then converted into electrical signals. These signals are then operated on to produce cyan, magenta and yellow electrical signals. These signals are then transmitted to a thermal printer. To obtain the print, a cyan, magenta or yellow dye-donor element is placed face-to-face with an element. The two are then inserted between a thermal printing head and a platen roller. A line-type thermal printing head is used to apply heat from the back of the dye-donor sheet. The thermal printing head has many heating elements and is heated up sequentially in response to one of the cyan, magenta or yellow signals, and the process is then repeated for the other two colors. A color hard copy is thus obtained which corresponds to the original picture viewed on a screen. Further details of this process and an apparatus for carrying it out are contained in U.S. Pat. No. 4,621,271.

Recently thermal dye transfer printing techniques have been applied to packaging materials such as pressure sensitive labels, glue applied labels, flexible packaging materials and wrapping materials. Thermal dye transfer printed packaging materials have been found to provide excellent image quality and are well integrated into a digital printing work flow were computer files are rendered and thermal printed into packaging substrates. Since packaging materials are widely handled by consumers and often are utilized in sterile environments such as a hospital or culture lab, there remains a need to incorporate antimicrobial materials into thermal printed packaging materials to reduce the probability of unwanted microbial activity.

Thermal transfer image receiving sheets for labels or stickers are known in the art including, for example, U.S. Pat. No. 6,153,558; U.S. Pat. No. 6,162,517; and U.S. Pat. No. 4,984,823. U.S. Pat. No. 6,162,517 to Oshima et al., for example, discloses a label comprising, disposed between a dye receptor layer and an adhesive layer, a foamed resin film layer and a non-foamed resin film layer. A bonding layer can be disposed between the foamed and non-foamed layers. U.S. Pat. No. 4,984,823 to Ishii et al. discloses, a label portion comprising an image-receiving layer, a sheet substrate, and an adhesive layer. The sheet substrate can be a resin film such as foamed polyethylene terephthalate, synthetic paper, and the like.

### PROBLEM TO BE SOLVED BY THE INVENTION

There remains a need to control the release of an antimicrobial active material from a high quality, thermal dye transfer printed packaging materials, such that a minimum inhibitory concentration of the antimicrobial metal may be achieved at the surfaces of the packaging material for the duration of the use of packaging material.

### SUMMARY OF THE INVENTION

It is an object of the invention to provide thermal dye transfer printed packaging materials having antimicrobial properties.

It is another object to provide a durable thermal dye transfer printed packaging materials.

It is a further object to provide an antimicrobial gradient on the surface of thermal printed packaging materials.

These and other objects of the invention are accomplished by a packaging material comprising a substrate, an image formed by thermal dye transfer on said substrate and a transparent polymer over layer on the opposite side of the image from said substrate, and further comprising antimicrobial composition in said over layer.

### ADVANTAGEOUS EFFECT OF THE INVENTION

The invention provides thermal dye transfer printed packaging substrates having antimicrobial properties. In one preferred embodiment, the invention provides a thermal transfer donor element having both protection properties and antimicrobial properties.

### DETAILED DESCRIPTION OF THE INVENTION

This invention provides a useful antimicrobial composition suitable for many packaging uses and particularly for the food industry, health and beauty, beverage and medical packaging. In addition, polymers that may be utilized in the antimicrobial composition are on the approved food contact list published by the Food and Drug Administration (Sec 177.1360). The composition of the invention quickly provides a minimum inhibitory concentration of the antimicrobial metal at the surface of the packaging substrate containing the antimicrobial composition under the common operating environments typical of packaging materials. The invention provides this effect for a sustained period of time even at relatively low lay downs of silver ion which are environmental safe and are cost effective compared to prior art methods of controlling microbial activity.

The invention provides thermally printed packaging substrates that are useful in digital printing workflows and provides excellent image quality, excellent text quality and provides both antibacterial and anti-fungal protection properties. Thermal printed packages have been shown to have high consumer impact, have lower printed inventories and can be printed on demand with variable data such as a patient name or changeable ingredient list. Thermally printed labels have value for security systems such as photo ID and security badges. These thermal printed packaging substrates have value in sterile environments such as hospitals, culture labs and food packaging. Further, by providing packaging substrates with antimicrobial properties, the spread of harmful active microbial contamination from commonly handled consumer goods such as hand soap containers; food packages and beverage containers can be reduced. In addition, the thermal printing of the packaging substrates provides rapid printing of packaging materials as digital files are quickly and efficiently rendered and printed compared to traditional printing presses. Also, thermal printing does not require undesirable solvents typically utilized in the printing industry, therefore the thermal printer can be located at or near packaging processes without solvent contamination of the packaged product.

Thermal printers typically print from a thin polymer donor element that is coated with thermal transfer dyes. Upon controlled heating of the donor element, the thermal dyes sublimate and transfer to packaging substrates. Further, prior art thermal imaging systems typically utilize a donor web containing a thin, transparent polymer capable of protecting thermally printed images. The invention allows for thermally transferable donor element capable of simultaneously protecting the delicate packaging substrate indicia and providing antimicrobial properties all in one printing step significantly simplifying the packaging printing process. The donor element containing the antimicrobial materials can be precisely thermally transferred to the surface of the packaging substrate, where the antimicrobial is most effective at reducing microbial activity. The amount of antimicrobial material transferred can be varied and be applied from the antimicrobial donor web by adjusting the amount of antimicrobial material to be printed. The donor element can also transfer the antimicrobial materials pattern-wise or image-wise allowing for precision application of the antimicrobial materials or aligning the antimicrobial materials with an image, text to form an antimicrobial area of the packaging substrate. In addition, the antimicrobial materials can be applied to the packaging substrate in a gradient, concentrating the antimicrobial materials in areas that require higher concentration such as the label area of a hand soap container. These and other advantages will be apparent from the detailed description below.

The terms as used herein, “top”, “upper”, “image side”, and “face” mean the side or toward the side of a dye image receiver sheet bearing the dye-receiving imaging layers. The terms “bottom”, “lower side”, and “back” mean the side or toward the side of the dye image receiver sheet opposite from the side bearing the dye imaging layers. The term used herein “peelable adhesive” or “repositionable adhesive” means an adhesive material that has a peel strength less than 100 grams/cm. The term used herein “permanent adhesive” means as adhesive materials that has peel strength of greater than 100 grams/cm. The term used herein “packaging substrate” or “base” or “support” means web materials that are commonly utilized in the packaging industry for protecting, storing and labeling packages. Examples of useful packaging substrates include paperboard, fabric, cardboard, pressure sensitive labels, glue applied labels, flexible packaging material, stand-up pouches and oriented polymer films. The term as used herein, “transparent” means the ability to pass visible radiation energy without significant deviation or absorption. For this invention, “transparent” material is defined as a material that has a spectral transmission greater than 85%.

The term used herein “dye donor element sticking” means the tendency of dye donor elements, which typically are thermal dyes coated onto thin oriented polymer, to stick to the dye receiver element. Dye donor element sticking is typically measured by printing high density color patches and making visual observations of the dye donor element sticking to the receiving layer. At the onset of sticking, vertical density lines, sometimes referred to as chatter, are observed down the printed page at a repeatable frequency. As used herein, the term “dye uptake” means the ability of any dye-receiving layer to accept dyes that are printed or thermally transferred. Dye uptake is typically related to the thermal printing temperature, chemistry of the dye-receiving layer, and chemistry of the dyes and the Tg of the dye-receiving layer. As used herein, the term “dye migration” means the tendency of the dyes to move in the dye-receiving layer after printing. Dyes that have a high amount of migration will result in an image becoming fuzzy, less sharp or text becoming fuzzy or the inability of bar code reading equipment to read printed black bar codes. Dye migration is typically related to ambient temperature, dye-receiving layer chemistry, Tg of the dye-receiving layer and amount of plasticizer in the dye-receiving layer.

Articles having antimicrobial properties may be prepared by application of an antimicrobial compound (hereafter referred to as AMC) to the surface of the article, or by embedding an AMC within the article. In most instances, bacteria, microbes or fungi may reside only at the surface of an article, and thus the AMC is applied only to the surface. The AMC may be applied by many methods such as coating, spraying, casting, blowing, extruding, etc. Typically, the AMC is dissolved or dispersed in a vehicle (such as a solvent) and a binder (such as a polymer), which provides a means of adhering the AMC to the article surface. The AMC can be incorporated within plastics and polymers to provide antibacterial and/or anti-fungal protection to the plastics and polymers in a variety of end-use applications. Incorporation of this material into a plastic or polymer is accomplished through the design and manufacture of a master batch, containing an elevated level of the active ingredient in a particulate form, and may include other ingredients that act to provide stability to the particulate form. The active ingredient can be incorporated into polypropylene, polyethylene, polyester, nylon, and other common polymers and plastics. The mixture subsequently melted and extruded to form a film. The film may then be attached to an article by means such as gluing or lamination.

Upon use and exposure of an antimicrobial article to conditions under which microbial growth may occur, the AMC (or in the case of an antimicrobial metal ion exchange material, the antimicrobial metal ion) may then leach from the surface of the article to kill or inhibit the growth of microbes present thereon. In order for the article to have antimicrobial properties, the AMC must leach out at a rate fast enough to establish and maintain a minimum inhibitory concentration (MIC). Below the MIC, microbial growth may continue uninhibited. Likewise, it is important that the AMC not leach out so fast as to quickly deplete the article of AMC and thus limit the longevity of the effectiveness of the article. The rate at which the AMC may leach (or diffuse) is dependent upon its degree of solubilization in aqueous media (water). This is an essential point, since microbial growth requires high water activity commonly found in wet or humid environments. Because most antimicrobial materials are substantially soluble in water, the rate of diffusion of the AMC will be limited by the rate at which water can diffuse to the AMC and hence dissolve it. This is especially true for solid-phase AMC's, since diffusion may not occur until the AMC is dissolved or solubilized. If the AMC is embedded in a polymer which very quickly adsorbs water, the article may be quickly depleted of antimicrobial activity, since the AMC contained at its surface may quickly leach into the surrounding environment. Alternatively, if the AMC is embedded in a polymer which does not adsorb water, or only adsorbs water extremely slowly, then the AMC may diffuse very slowly or not at all, and a MIC may never be achieved in the surrounding environment. A measure of the permeability of various polymeric addenda to water is given by the permeability coefficient, P, which is given by
P=(quantity of permeate)(film thickness)/[area×time×(pressure drop across the film)]
Permeability coefficients and diffusion data of water for various polymers are discussed by J. Comyn, in Polymer Permeability, Elsevier, NY, 1985 and in “Permeability and Other Film Properties of Plastics and Elastomers,” Plastics Design Library, NY, 1995. The higher the permeability coefficient, the greater the water permeability of the polymeric media. The permeability coefficient of a particular polymer may vary depending upon the density, crystallinity, molecular weight, degree of cross-linking, and the presence of addenda such as coating-aids, plasticizers, etc.

The composition utilized in the invention comprises an antimicrobial compound in a polymer matrix or overlay that both serves to provide antimicrobial properties and provide protection to the thermally printed layer from the rigors of packaged materials such as abrasion, elevated temperature, high humidity and freezer consitions. Preferably the polymer matrix comprises an antimicrobial compound and a polyethylene-polyvinylalcohol copolymer, wherein the antimicrobial compound is embedded in the copolymer. Either the compound itself or an antimicrobial moiety released from the antimicrobial compound is preferably aqueously soluble. The polyethylene-polyvinylalcohol copolymer is preferred because its water permeability is intermediate and thus it allows for facile diffusion of the AMC contained within, to the surface of an article. This allows for a MIC to be achieved at the surface without quickly depleting the article of all AMC. Thus, the antimicrobial properties of the article are long-lived. The polyethylene-polyvinylalcohol co-polymer may also serve as a binder to allow for adhesion of an AMC to a surface, article, or substrate. The fraction of polyvinyl alcohol in the copolymer should be from about 20% to 80%, and more preferably from about 45% to 75%. The copolymer may have a wide range of molecular weight, but it is preferred that the copolymer have an average molecular weight between 100,000 and 1,000,000. It is preferred that the water permeability coefficient of the polyethylene-polyvinylalcohol copolymer be from about 5000 to 15000 [(cm³ cm)/(cm² sec/Pa)]×10¹³.

To form the inventive composition, the antimicrobial compound should be uniformly and homogeneously mixed within the polyethylene-polyvinylalcohol copolymer. Mixing may be accomplished by a number of methods. For example, the copolymer and the AMC may be dispersed in a suitable solvent and then coated or dried to form a solid mixture. Typically, the solvent will be an alcohol/water mixture. The process may include the addition of surfactants, peptizers, dispersion aids, etc. to facilitate the mixing. Alternatively the mixture may be formed by directly compounding the polymer and AMC at the melting temperature of the polymer as is done by screw compounding.

The antimicrobial active compound of the antimicrobial composition may be selected from a wide range of known antibiotics and antimicrobials. Suitable materials are discussed in “Active Packaging of Food Applications” A. L. Brody, E. R. Strupinsky, and L. R. Kline, Technomic Publishing Company, Inc. Pennsylvania (2001). Examples of antimicrobial agents suitable for practice of the invention include benzoic acid, sorbic acid, nisin, thymol, allicin, peroxides, imazalil, triclosan, benomyl, antimicrobial metal-ion exchange material, metal colloids, metal salts, anhydrides, and organic quaternary ammonium salts.

In a preferred embodiment, the antimicrobial compound is selected from metal ion-exchange materials which have been exchanged or loaded with antimicrobial ions. Metal ion-exchange materials suitable for practice of the invention are selected from zirconium phosphates, metal hydrogen phosphates, sodium zirconium hydrogen phosphates, zeolites, clays such as montmorillonite, ion-exchange resins and polymers, porous alumino-silicates, layered ion-exchange materials, and magnesium silicates. Preferred metal ion exchange materials are zirconium phosphate, metal hydrogen phosphate, sodium zirconium hydrogen phosphate, or zeolite. Preferred antimicrobial ions are silver, copper, nickel, zinc, tin, and gold. In a particularly preferred embodiment the antimicrobial ions are selected from silver and zinc. The silver maybe in the form of silver halide particles which can be of any shape and halide composition. The type of halide can include chloride, bromide, iodide and mixtures of them. The silver halide particles can include, for example, silver bromide, silver iodobromide, bromoiodide, silver iodide or silver chloride. However, the embodiment is not limited to these compositions, and any suitable composition can be used. In one embodiment, the silver halide particles are predominantly silver chloride. The predominantly silver chloride particles can include, but is not limited to, silver chloride, silver bromochloride, silver iodochloride, silver bromoiodochloride and silver iodobromochloride particles. By predominantly silver chloride, it is meant that the particles are greater than about 50 mole percent silver chloride. Preferably, they are greater than about 90 mole percent silver chloride, and optimally greater than about 95 mole percent silver chloride. The silver halide particles can either be homogeneous in composition or the core region can have a different composition than the shell region of the particles. The shape of the silver halide particles can be cubic, octahedral, tabular or irregular. More silver halide properties can be found in “The Theory of the Photographic Process”, T. H. James, ed., 4th Edition, Macmillan (1977). In another embodiment the silver halide particles have a mean equivalent circular diameter of less than 1 micron, and preferably less 0.5 microns.

The antimicrobial ion is the antimicrobial moiety of the antimicrobial compound. In yet another preferred embodiment the antimicrobially active compound is represented by the general formula:
M(H_{1-x-y}NaₓAg_{y}PO₄)₂.H₂O;
wherein M=Ti and Zr and x and y are greater than zero and less than one. An example preparation of this material is given in the example section, and the preparation of these material are discussed at length in U.S. application Ser. No. 10/324,234 85124 filed Dec. 19, 2002.

The antimicrobial compound, particularly an antimicrobial metal ion exchange material, is preferably 0.1 to 5.0% by weight of the composition. It is preferred, when the antimicrobial ion is silver, that the silver ion comprises 0.01 to 1.0% by weight of the composition.

The composition utilized in the invention may be applied to the surfaces of thermally printed packaging substrates to prevent the growth of microbes such as bacteria, mold, and yeast and to reduce the risk of the transmission of infectious disease. The inventive composition may be applied to the thermally printed packaging substrate by many known methods such as spraying, molding, gravure coating, blade coating and extruding, etc. Alternatively, the inventive coating may be applied to a substrate as a plastic film and the film adhered to the thermally printed packaging substrate by means of post printing lamination or adhesive lamination.

Pressure sensitive labeling of packaging substrates is a very popular prior art method for the decoration of packages. Pressure sensitive labels provide an excellent opportunity for thermal printing as thermally printed graphics, text and images are of high quality compared to flexography or gravure printing. Thermally printed pressure sensitive label having antimicrobial properties, preferably comprise a pressure sensitive adhesive typically located on the side opposite the printed image. Organic pressure sensitive adhesives may be natural or synthetic. Examples of natural organic pressure sensitive adhesives include bone glue, soybean starch cellulosics, rubber latex, gums, terpene, mucilages and hydrocarbon resins. Examples of synthetic organic pressure sensitive adhesives include elastomer solvents, polysulfide sealants, thermoplastic resins such as isobutylene and polyvinyl acetate, thermosetting resins such as epoxy, phenoformaldehyde, polyvinyl butyral and cyanoacrylates and silicone polymers. For single or multiple layer pressure sensitive adhesive systems, the preferred pressure sensitive adhesive composition is selected from the group consisting of natural rubber, synthetic rubber, acrylics, acrylic copolymers, vinyl polymers, vinyl acetate-, urethane, acrylate-type materials, copolymer mixtures of vinyl chloride-vinyl acetate, polyvinylidene, vinyl acetate-acrylic acid copolymers, styrene butadiene, carboxylated styrene butadiene copolymers, ethylene copolymers, polyvinyl alcohol, polyesters and copolymers, cellulosic and modified cellulosic, starch and modified starch compounds, epoxies, polyisocyanate, polyimides.

In order to provide additional antimicrobial protection to the thermally printed pressure sensitive labels, the antimicrobial materials are also preferably added to the pressure sensitive adhesive which adds additional antimicrobial protection to the adhesive. The addition of the antimicrobial materials to the pressure sensitive adhesive are desirable for application requiring pressure sensitive adhesive to human skin contact. The addition of the antimicrobial materials allow for thermally imaged substrates to be applied to the surface of human skin reducing the tendency of prior art pressure adhesives to create a favorable environment for the growth of unwanted microbes. Examples include trans-dermal patches for nicotine dispensing or contraceptive dispensing. Other examples include body art, fingernail decorations and costumes. Additionally, the use of aqueous pressure sensitive adhesive formulations, which eliminate the need for solvent emissions, can be a medium for unwanted microbial activity. The preferable addition of the antimicrobial materials to both the overlay and the adhesive has been shown to significantly reduce antimicrobial activity particularly for labels that are exposed to both high temperature and high humidity.

The invention provides a thermally printed packaging substrate containing a transparent polymer overlay having antimicrobial properties. In one embodiment of the invention, the overlay comprises more than one layer. Additional layers can provide features important to packaging substrates such as oxygen barrier, vapor barrier, puncture resistance, antistatic properties, electrical conductivity and the like. An example of a multiple layered overlay is a follows:

Polymer overlay containing AMC

Polymer overlay containing AMC and vapor barrier

Thermally printed dye receiver layer

Packaging substrate

In a further embodiment of the invention, the overlay containing antimicrobial materials is preferably printed in a pattern by known methods such as ink jet printing, gravure printing or thermal printing. The pattern can be applied image-wise or pattern-wise and can be applied to specific areas of the thermally printed substrate. The pattern may also contain roughness, preferably greater than 5 micrometers, to increase surface area of the exposed antimicrobial materials and provide texture to the packaging substrate.

In a suitable embodiment the antimicrobial layer has a thickness in the range of 0.1 μm to 100 μm, and more preferably the thickness of said antimicrobial layer is about 1 μm to 10 μm. Generally the substrate has a thickness in the range of 0.025 mm to 5 mm. In a preferred embodiment utilizing an antimicrobial ion exchange material, wherein silver is the antimicrobial ion, the silver lay down is preferably from 1 mg/m² to 1000 mg/m². The medium may then be attached to the surface of an article to impart antimicrobial activity to that item. The antimicrobial layer should be placed such that it is the outermost surface of the article to maximize its antimicrobial activity. The medium may be attached by any means such as lamination, gluing, wrapping, etc.

In the practice of the invention, a vehicle may be used to facilitate adhesion or application of the inventive composition or inventive medium to a surface, a fabric, or article to impart antimicrobial activity to that item. The vehicle serves multiple purposes including aiding the application of the antimicrobial composition via painting, spraying, coating, etc, binding the antimicrobial to that surface, and preventing the loss of antimicrobial activity due to normal wear or use. The vehicle used may be a polymer, a polymeric latex, a polymeric resin, an adhesive, or a glass or ceramic vehicle; i.e., the vehicle should comprise no more than 40% of the vehicle/antimicrobial composition mixture.

In order to provide a high quality thermal dye transfer printed packaging substrate, it is desirable for the packaging substrate to contain a dye receiving layer for efficient high quality printing and to reduce dye mobility which would reduce the quality of the printed substrate. In order to provide a dye-receiving layer that is capable of efficiently receiving dyes and avoid the need for expensive and problematic lubrication chemistry a dye image receiver sheet comprising a dye-receiving layer comprising a cross-linked copolymer of polyester and a lubricator polymer, wherein said polyester component of said cross-linked copolymer is present in an amount of between 75% and 99% by weight is preferred. The polyester component of the copolymer of the invention provides excellent uptake of dye and excellent dye retention. The lubricator component of the copolymer provides lubrication to resist sticking of dye donor web materials at the pressures and temperatures common during thermal dye transfer. Since the polyester component provides the dye uptake and retention properties, the polyester component of the copolymer is the majority component. Polyester component below 70% by weight of copolymer, the dye uptake and dye retention are reduced to an unacceptably low level, reducing the quality of the printed image. Above, 99.5% by weight of copolymer, little lubrication is provided to thermal dye transfer donor webs, significantly increasing donor web sticking to the receiving layer. A cross-linked copolymer of polyester and lubricator polymer is preferred because cross-linking the copolymer of the invention improves web adhesion, aids in coating and subsequent drying of the coated dye-receiving layer and improves the mechanical properties of the coated, dried dye-receiving layer.

The dye receiver layer of the invention preferably comprises a plasticizer. Plasticizer addition to the dye receiver layer has been shown to increase the dye uptake while not significantly increasing dye donor element sticking during thermal dye transfer. The preferred plasticizer addition by weight of the copolymer is between 1 and 5% by weight. Above 10% addition plasticizer has been shown to significantly increase dye migration in the printed image, which renders the image fuzzy and lower dye density. Preferred plasticizers utilized in the dye receiver layer utilized in the invention are aliphatic esters and phthalate esters.

The dye receiver layer is preferably capable of forming a thermal image that has a maximum cyan, magenta, and yellow formed black density of greater than 2.0. A black density of less than 1.8, while allowing for a good quality image tends to be viewed as low quality for packaging materials such as pressure sensitive labels, flexible packaging and stand-up pouches. In packaging applications, bar codes are important to retail. Bar codes with black density less than 1.8 are difficult to read and can result in accounting errors during scanning of bar codes. Black dye density is measured on a Status A reflection densitometer. Maximum dye density is created when maximum amounts of yellow, magenta and cyan dyes have been transferred in registration to a 4 cm² patch in the receiver layer.

The dye receiver layer applied to the surface of the substrate preferably has a roughness average less than 3.0 micrometers. A smooth dye receiver layer is essential to the quality of a thermal dye transfer image. By providing a dye receiver layer with a roughness average less than 3.0, unwanted image drop-outs caused by uneven contact between the dye donor element and the receiver layer are not formed. Roughness average of the dye receiver layer is measured by TAYLOR-HOBSON Surtronic 3 with 2 micrometers diameter ball tip. The output Ra or “roughness average” from the TAYLOR-HOBSON is in units of micrometers and has a built in cut off filter to reject all sizes above 0.25 mm.

Lubricator polymers utilized in the invention provide lubrication between the cross-linked dye receiver layer and dye donor elements such as 6 micrometer PET. During thermal dye transfer printing of images, test or graphics, a resistive thermal head is brought into contact with dye donor element. Dye is transferred to the dye-receiving layer by thermal heat generated by the resistive head and pressure between the resistive thermal head and the dye-receiving layer. Preferred lubrication polymers, which are in a copolymer with polyester, provide the desired lubrication. In an embodiment of the invention, polyurethane polymer is preferred for a lubrication polymer. Polyurethanes are formed by reacting a polyol with a diisocyanate or a polymer isocyanate in the presence of suitable catalysts and additives. Polyurethane in a copolymer with polyester has been found to provide donor element lubrication during thermal dye transfer, can be formed into a copolymer with polyester, does not interfere with the formation of the dye based image and has design flexibility to provide a target dye receiver layer Tg for high printed dye density. Further, a polyester-based polyurethane polymer achieves a particular balance of strength and flexibility that is desirable for a dye receiving layer. For polyester-based polyurethane polymers useful in the present invention, convenient measures of the strength and flexibility attributes are 100% modulus as an indicator of strength and percent elongation to break as an indicator of flexibility. 100% modulus is defined as the tensile strength measured at 100% elongation and is measured utilizing ASTM D 638. 100% modulus is preferably in the range of 27 to 41 MPa. Elongation to break is preferably in the range of 150-300% and is measured utilizing ASTM D 638.

The polyester-based polyurethane polymer may be made from a variety of polyester polyols and polyisocyanates. When made from difunctional polyester polyols (2 hydroxyl groups per polyester polyol molecule) and diisocyanates, the polymer is typically made by preparing a prepolymer at a stoichiometric ratio of isocyanate groups to hydroxyl groups (NCO/OH ratio) of greater than one, preferably in the range of from 1.3 to 3.0 and optimally in the range of from 1.5 to 2.7. Mixtures of polyols and mixtures of polyisocyanates may be used and it is possible to include other polyfunctional reactive nucleophiles, and also polyols and/or polyisocyanates with functionalities greater than 2. If polyols or polyisocyanates of functionality different than 2 are employed it is especially necessary to control the amounts of reactants having functionality different than 2 and to adjust NCO/OH so as to avoid either excessive chain termination or extensive network formation that could lead to gelation of the pre-polymer.

To aid in dispersibility in water, groups that are hydrophilic, or that can be converted to hydrophilic groups, are customarily chemically incorporated into the pre-polymer. Typical of hydrophilic groups are backbone constituents with pendant polyethylene oxide chains. These act as nonionic stabilizing groups. Commonly used to create anionic stabilizing groups are carboxylic acid or sulfonic acid groups that hang off the prepolymer backbone. These become hydrophilic after salting them with tertiary amines, or the inverse can be done, where backbone or pendant tertiary amino groups can be salted with acids, giving rise to cationic stabilization. However made, the prepolymeric, isocyanate-terminated intermediate is typically dispersed in water or water containing one or more surfactants and right after dispersion is chain extended by reaction of remaining, unreacted isocyanate groups with polyfunctional nucleophiles. When salting is used for stabilization, the prepolymer can be salted before it is dispersed, or the salting amine or acid as the case may be can be placed in the water phase before dispersion. Chain extension increases molecular weight and affords an aqueous dispersion of a polymeric urethane. The chain extender is a di or polyfunctional reactive nucleophile that reacts with unreacted isocyanate groups. Chain extender to unreacted isocyanate group stoichiometry is usually chosen to maximize molecular weight of the polyurethane. The reactive nucleophile groups in the chain extender can be amino (including hydrazine), hydroxyl, or other reactive groups. Even water can function as a chain extender. Mixtures of chain extenders, or chain extenders with more than one kind of reactive nucleophilic group, for example, an aminoalcohol, can be used.

While polyurethane has been shown to be an excellent lubricator polymer for thermal dye transfer printing of the dye receiver layer and provide a compliant layer adjacent to dye donor elements, other copolymers may be suitable to provide both good dye uptake while reducing dye donor element sticking. Other suitable polyester copolymers for thermal dye transfer printing include polycarbonate, polycyclohexylenedimethylene terephthalate and vinyl modified polyester copolymers.

The glass transition temperature or Tg of the cross-linked dye receiver layer is an important determining factor in the dye density of the printed image. A high dye-receiving layer Tg tends to have low dye uptake but very low dye donor element sticking. A low dye-receiving layer Tg tends to have high dye uptake but very high levels of unwanted dye donor element sticking. Tg is conveniently measured utilizing the well known measurement technique known as DSC. The preferred dye-receiving layer is between 42 and 72 degrees Celsius, more preferably between 42 and 62 degrees C. A dye-receiving layer having a Tg below 40 degrees C. has been shown to exhibit dye donor sticking. A dye-receiving layer having a Tg greater than 75 degrees C. does not allow the dyes to migrate into the dye receiver layer resulting in low image density. The range of 42 to 62 degrees C. has been found to provide both excellent dye uptake in the cross-linked copolymer of the invention and dye donor element sticking performance utilizing resistive head thermal printers. Most preferably, the Tg of the dye-receiving layer of the invention is about 52 degrees Celsius. Since the measurement of Tg typically contains measurement error of about 2% and manufacturing variability can contribute another 3% of variation, there exist some acceptable range around a Tg of 52 degrees Celsius, hence the term about 52 degrees Celsius.

Cross-linking of the polyester/lubricator copolymer is preferred and has been shown to improve the mechanical properties of the dye receiver layer, improve adhesion to oriented polymer webs compared to polyester/lubricator polymers without a high degree of cross-linking and allow for good film formation during coating of the dye receiver layer. In a preferred embodiment, the lubricator polymer comprises polyurethane and the cross-linking material comprises trimethylolpropane tris(2-methyl-1-aziridine propionate) present in amount of between 0.20 and 0.85 weight % of the cross-linked polymer. Trimethylolpropane tris(2-methyl-1-aziridine propionate) has been shown to be an effective cross-linking material for a polyester/polyurethane copolymer and provides good dye uptake.

One of the many benefits of the cross-linked copolymer dye receiver layer is an improvement is scratch resistance of the printed dye receiver layer. Scratch resistance is particularly important during the handling of images or for packaging materials that must withstand the rigors of a packaging operation. The cross-linked copolymer of the invention preferably has a scratch resistance of between 0.1 and 1.0 mN. Scratch resistance is measured by dragging a steel tip with a radius of 5 micrometer across the dye receiver layer at a rate of 10 cm/min. The steel tip is progressively loaded until scratching in the dye receiver layer is first observed. The load for which a scratch in the dye receiver layer is first observed is the recorded load. A scratch resistance less than 0.08 scratches too easily and can easily be damaged during handling of the printed dye receiver image. A scratch resistance greater than 1.1 mN has been shown to unacceptably reduce dye uptake because a dye receiving layer with a scratch resistance greater than 1.1 mN is hard and difficult for the dye to migrate into under typical thermal dye transfer printing.

In another preferred embodiment, the antimicrobial materials are preferably are present in both the polymer overlay layer and the dye receiving layer. By providing the antimicrobial materials in both the polymer overlay and the dye receiving layer, the antimicrobial properties of the thermally printed packaging substrate are further enhanced. This is particularly important for aqueous dye receiving layers, which have a tendency to provide the proper medium for microbial activity. Aqueous dye receiver layers are environmentally friendly and are particularly well suited for food contact. The addition of the antimicrobial materials to the dye receiving layers allows for the use of the desirable aqueous dye receiver chemistry while reducing the tendency of the aqueous layer to support microbial activity. Examples include wine bottle labels, labels used in high humidity regions such as South-east Asia, beverage containers, archival labels.

Since the printing process required web materials to be wound and unwound, the opportunity to generate a static charge on one or more of the webs materials is present. In a preferred embodiment of the invention, the dye-receiving sheet of the invention contains an antistatic material and preferably has a resistivity of less than 10¹¹ ohms/square. A wide variety of electrically-conductive materials can be incorporated into adhesive layers and/or dye-receiving layers to produce a wide range of conductivities. These can be divided into two broad groups: (i) ionic conductors and (ii) electronic conductors. In ionic conductors charge is transferred by the bulk diffusion of charged species through an electrolyte. Here the resistivity of the antistatic layer is dependent on temperature and humidity. Antistatic layers containing simple inorganic salts, alkali metal salts of surfactants, ionic conductive polymers, polymeric electrolytes containing alkali metal salts, and colloidal metal oxide sols (stabilized by metal salts), described previously in patent literature, fall in this category. However, many of the inorganic salts, polymeric electrolytes, and low molecular weight surfactants used are water-soluble and are leached out of the antistatic layers during processing, resulting in a loss of antistatic function. The conductivity of antistatic layers employing an electronic conductor depends on electronic mobility rather than ionic mobility and is independent of humidity. Antistatic layers which contain conjugated polymers, semi-conductive metal halide salts, semi-conductive metal oxide particles, etc. have been described previously. In the most preferred embodiment, the antistat material comprises at least one material selected from the group consisting of tin oxide and vanadium pentoxide.

In another preferred embodiment of the invention antistatic material are incorporated into the pressure sensitive adhesive layers. The antistatic material incorporated into the pressure sensitive adhesive layer provides beneficial static reduction between the dye receiving layer and dye donor elements. Further the antistatic material reduces the static on the label which has been shown to aid labeling of containers in high speed labeling equipment. As a stand-alone or supplement to the carrier comprising an antistatic layer, the pressure sensitive adhesive may also further comprise an antistatic agent selected from the group consisting of conductive metal oxides, carbon particles, and synthetic smectite clay, or multi-layered with an inherently conductive polymer. In one of the preferred embodiments, the antistatic material is metal oxides. Metal oxides are preferred because they are readily dispersed in the thermoplastic adhesive and can be applied to the polymer sheet by any means known in the art. Conductive metal oxides that may be useful in this invention are selected from the group consisting of conductive particles including doped-metal oxides, metal oxides containing oxygen deficiencies, metal antimonates, conductive nitrides, carbides, or borides, for example, TiO₂, SnO₂, Al.₂O₃, ZrO₃, In₂O₃, MgO, ZnSb₂O₆, InSbO₄, TiB₂, ZrB₂, NbB₂, TaB₂, CrB₂, MoB, WB, LaB₆, ZrN, TiN, TiC, and WC. The most preferred materials are tin oxide and vanadium pentoxide because they provide excellent conductivity and are transparent.

The receiver sheet for the element of the invention may be transparent or reflective, and may be a polymeric, a synthetic paper, or a cellulosic paper support, or laminates thereof. In a preferred embodiment, a cellulose paper support is used. In a further preferred embodiment, a polymeric layer is present between the paper support and the dye image receiving layer. For example, there may be employed a polyolefin such as polyethylene or polypropylene. In a further preferred embodiment, white pigments such as titanium dioxide, zinc oxide, etc., may be added to the polymeric layer to provide reflectivity. In addition, a subbing layer is preferably utilized over this polymeric layer in order to improve adhesion to the dye image-receiving layer. In particular, oriented polymer sheets that have low surface energy such as polypropylene can be improved for dye receiver layer adhesion with the use of a subbing layer. Suitable subbing layers for dye receiving layer adhesion to polymeric web materials are disclosed in U.S. Pat. Nos. 4,748,150; 4,965,238; 4,965,239; and 4,965,241.

In another preferred embodiment of the invention, the substrate comprises an oriented polymer. Oriented polymers tend to be thin, strong and smooth sheets that have been shown to be excellent substrates for the dye receiver layer of the invention. Further, dye receiver layer coated oriented polymer sheets can be utilized for packaging applications such as stand-up pouches and snack food packaging. Oriented polymer sheets coated with the dye-receiving layer of the invention can also be used as point of purchase display and signs.

Thermal dye transfer imaging technology can simultaneously print text, graphics, and photographic quality images on the pressure sensitive label. Since the thermal dye transfer imaging layers of the invention are both optically and digitally compatible, text, graphics, and images can be printed using known digital printing equipment such as lasers and CRT printers. Because the thermal dye transfer system is digitally compatible, each package can contain different data enabling customization of individual packages without the extra expense of printing plates or cylinders. Further, printing digital files allows the files to be transported using electronic data transfer technology such as the Internet thus reducing the cycle time to apply printing to a package. Thermal dye transfer imaging layers allow competitive printing speeds compared to current ink jet printing methods.

The addition of a fiducial mark to the thermal dye transfer formed image is preferred as the fiducial mark provides a means for die cutting the image to create a label. The addition of a fiducial mark allows the article to be die cut using optical sensors to read the registration of the image. The fiducial mark may be printed on the base material, printed using thermal dye transfer formed images or post process printed using printed inks. In another embodiment, the fiducial mark is created utilizing a mechanical means such as punched hole, mechanical embossing or a partial punched hole to create a topographical difference in the thermal dye transferred formed image. A mechanical fiducial mark allows for mechanical sensors to be used for die cutting, application of a spot printed color or for locating a label on a package during automated labeling.

Dye-donor elements that are used with the element of the invention conventionally comprise a support having thereon a dye containing layer. Any dye can be used in the dye-donor employed in the invention, provided it is transferable to the layer by the action of heat. Especially good results have been obtained with sublimable dyes. Dye donors applicable for use in the present invention are described, e.g., in U.S. Pat. Nos. 4,916,112; 4,927,803; and 5,023,228. As noted above, dye-donor elements are used to form a dye transfer image. Such a process comprises image-wise-heating a dye-donor element and transferring a dye image to an element as described above to form the dye transfer image. In a preferred embodiment of the thermal dye transfer method of printing, a dye donor element is employed which compromises a poly(ethylene terephthalate) support coated with sequential repeating areas of cyan, magenta, and yellow dye, and the dye transfer steps are sequentially performed for each color to obtain a three-color dye transfer image. When the process is only performed for a single color, then a monochrome dye transfer image is obtained.

Thermal printing heads, which can be used to transfer dye from dye-donor elements to receiving elements of the invention, are available commercially. There can be employed, for example, a Fujitsu Thermal Head (FTP-040 MCS001), a TDK Thermal Head F415 HH7-1089, or a Rohm Thermal Head KE 2008-F3. Alternatively, other known sources of energy for thermal dye transfer may be used, such as lasers as described in, for example, GB No. 2,083,726A.

A thermal dye transfer assemblage comprises (a) a dye-donor element, and (b) a element as described above, the element being in a superposed relationship with the dye-donor element so that the dye layer of the donor element is in contact with the dye image-receiving layer of the receiving element.

When a three-color image is to be obtained, the above assemblage is formed on three occasions during the time when heat is applied by the thermal printing head. After the first dye is transferred, the elements are peeled apart. A second dye-donor element (or another area of the donor element with a different dye area) is then brought in register with the element and the process repeated. The third color is obtained in the same manner.

Prior art donor elements typically comprise a thin polymer web coated with dyes that upon heating sublimate from the donor web to a receiving layer, forming an image. In a preferred embodiment of the invention, a thermally printed mass transfer donor element containing both a polymer layer suitable for protecting printed packaging materials and antimicrobial materials is utilize to simultaneously provide protection and antimicrobial properties to thermally printed packaging materials. Thermal mass transfer printing of the preferred donor element occurs as thermal energy causes the protective polymer containing the antimicrobial materials to “release” from the donor element and adhere to a dye receiving layer applied to the surface of packaging substrates. The amount of heat applied to the antimicrobial donor element is a factor in determining the rate of mass transfer, the amount of mass transfer and the bond strength between the dye receiving layer and the protective layer containing antimicrobial materials. A thermally printed protective, antimicrobial layer allows for the packaging substrate to be printed and protected with antimicrobial materials in one efficient printing step. By providing a antimicrobial thermal donor element, thermally printed packaging substrates of the invention can be easily printed with the desired packaging content and then subsequently printed with a protective polymer layer containing the preferred antimicrobial materials. An example of a cross section of a preferred donor element is as follows:

Transferable polymer containing antimicrobial materials

Oriented polymer film

Slip layer

A donor element for providing an antimicrobial layer overlaying an image comprising in order a slip layer, an oriented polymer film, and a thermally transferable polymer matrix containing antimicrobial composition is preferred. By providing a donor element suitable for thermal printing, packaging substrates can be dye printed and then over printed with a overlay polymer simultaneously protecting the thermally printed image and providing antimicrobial properties to the thermally printed image. The simultaneous application of the protective overlay and antimicrobial materials allows printing and protection to occur in one efficient step avoiding the need for an expensive and time consuming application of antimicrobial materials post printing. The slip layer is preferred to reduce the friction between the polymer film and the dye transfer head allowing for efficient printing, particularly on long printing runs when the thermal head can heat up to 70 degrees C. Preferably, the glass transition temperature (Tg) of the thermal dye receiving layer is less than the Tg of the thermally transferable polymer matrix.

In a preferred embodiment of the invention, the transferable polymer contains two or more layers. Two or more transferable polymer layers provide the ability to sub-optimize each of the layers for an intended purpose. For example, one polymer layer can be utilized to provide easy thermal separation from the oriented polymer film, while the other polymer layer can provide the antimicrobial materials. Another utilities of multiple layers include the addition of polymer layer(s) that provide vapor barrier, oxygen barrier, antistatic layer, anti-glare layer, polymer beads for a matte appearance, plasticizer containing layer, a second layer containing antimicrobial materials, electrically conductive layer and puncture resistance. A cross section of a preferred donor element having two layers is as follows:

Transferable polymer containing antistatic properties

Transferable polymer comprising antimicrobial materials

Oriented polymer film

Slip layer

In another embodiment of the invention, the transferable polymer layer contains heat expandable beads. By providing heat expandable beads, the transferable polymer containing the antimicrobial materials can be used to reduce the gloss of the thermal dye transfer image. Beads have also been shown to increase the amount of exposed surface area, thereby exposing more of the antimicrobial materials to surfaces that might contain unwanted active microbes. Heat expandable beads are known in the art and typically comprise polymer beads containing a gas such as butane. Upon thermal transfer, the gas in the beads expands and provides a surface texture.

In a further embodiment of the invention, the transferable polymer comprises a repeating pattern having a roughness of at least 5 micrometers. It has been found that by transferring a rough repeating pattern to the surface of a packaging substrate, the gloss of the thermal dye transfer printing is reduced and the antimicrobial materials have a higher exposed surface area compared to flat, planner surfaces. Examples of repeating patterns include sine functions, square waves, curved individual lenses, grid of intersecting lines and circular patterns. The desired patterns can be applied to the surface of the oriented polymer sheet at the time of manufacture and are transferred by typical thermal print heads to the surface of the packaging substrates.

In another embodiment of the invention, the oriented polymer film preferably contains an antimicrobial composition of the surface of the oriented polymer film. By providing the antimicrobial materials on the surface of the oriented polymer film, at the time of thermal transfer, it has been found that both the transferable polymer and the layer of antimicrobial material on the surface of the oriented polymer sheet thermally transfer. This has the advantage of providing a high concentration level of antimicrobial material on the surface of the transferable polymer thereby reducing the amount of antimicrobial material required and increase the exposure of the materials to the surrounding environment. A cross section of a preferred donor element having a high concentration of antimicrobial materials located at the surface of the oriented donor film is as follows:

Transferable polymer with Tg=68 degrees C.

Antimicrobial material

Oriented polymer film

Slip layer

In a further embodiment of the invention, the thermally transferable polymer preferably comprises indicia indicating the presence of the antimicrobial materials. This allows for transfer of the antimicrobial materials and a consumer indication that the materials have been applied. The presence can be indicated, for example, by the words “treated with antimicrobial materials” or “this surface is antimicrobial” or “clean spot” or the like. The presence can also be indicated by a color or pattern in the transferred polymer contain the antimicrobial materials. The reverse printing can be applied to the surface of the transferable polymer or to the oriented polymer web.

In another embodiment of the invention, the transferable polymer comprises colored materials. Colored materials such as dyes and pigments can provide a distinct color to the thermal mass applied protective layer. Further, the colored materials can be utilized to color correct for the native coloration of the antimicrobial materials or polymer materials allowing an image to be neutral or slightly blue for example. The addition of the colored materials can also be a signal to the consumer that the antimicrobial material are present on the thermally printed packaging materials or direct the consumers attention to a specific areas of the thermally printed packaging material that contains the antimicrobial materials.

What is claimed is:

1. A packaging material comprising a substrate, an image formed by thermal dye transfer on said substrate and a transparent polymer overlayer on the opposite side of the image from said substrate, and further comprising antimicrobial composition in said overlayer.

2. The packaging material of claim 1 wherein said substrate comprises a metallic layer.

3. The packaging material of claim 1 wherein said substrate comprises an oriented polymer.

4. The packaging material of claim 1 wherein said transparent polymer overlayer further comprises an anti-fugal material.

5. The packaging material of claim 1 further comprising pressure-sensitive adhesive on the side of said substrate opposite to said image.

6. The packaging material of claim 5 wherein said pressure-sensitive adhesive comprises an antimicrobial composition.

7. The packaging material of claim 1 wherein said overlayer comprises more than one layer.

8. The packaging material of claim 7 wherein the surface layer comprises hydrophilic polymer and microbial composition and a lower layer comprises hydrophobic polymer.

9. The packaging material of claim 1 wherein said overlayer is in a pattern.

10. The packaging material of claim 1 wherein said packaging material comprises a label.

11. The packaging material of claim 1 wherein said packaging material comprises a complete package covering.

12. The packaging material of claim 1 wherein said packaging material comprises a flexible pack.

13. The packaging material of claim 1 wherein said antimicrobial compound comprises silver halide

14. The packaging material of claim 1 wherein said packaging material comprises a wine label.

15. The packaging material of claim 1 wherein said packaging material comprises packaging for pharmaceutical applications.

16. The package material of claim 1 wherein said image formed by thermal dye transfer has a maximum cyan, magenta, and yellow formed black density of greater than 2.0.

17. The package material of claim 1 wherein said image is formed in a receiver layer comprising a cross-linked copolymer of polyester and polyurethane polymer, wherein said polyester component of said cross-linked copolymer is present in an amount of between 75% and 99% by weight.

18. The package material of claim 17 wherein said cross linked polymer was cross linked utilizing trimethylolpropane tris(2-methyl-1-aziridine propionate) in amount of between 0.20 and 0.85 weight % of the cross linked polymer.

19. The package material of claim 1 wherein said antimicrobial compound is benzoic acid, sorbic acid, nisin, thymol, allicin, peroxide, imazalil, triclosan, benomyl, antimicrobial metal-ion exchange material, metal colloid, anhydride, or organic quaternary ammonium salt.

20. The package material of claim 1 wherein said antimicrobial compound is an antimicrobial metal-ion exchange material which is a metal-ion exchange material which has been exchanged or loaded with antimicrobial ions.

21. The package material of claim 20 wherein said metal ion exchange material is zirconium phosphate, metal hydrogen phosphate, sodium zirconium hydrogen phosphate, zeolite, clay, an ion-exchange resin, an ion exchange polymer, porous alumino-silicate, a layered ion-exchange material, or magnesium silicate.

22. The package material of claim 1 wherein the antimicrobial compound is a silver ion exchange material; and wherein the polyethylene-polyvinylalcohol copolymer has a polyvinylalcohol content from about 25% to 35% by weight of the polyethylene-polyvinylalcohol copolymer, an average molecular weight of 100,000 to 1,000,000 and a water permeability coefficient of from 5000 to 15000 [(cm³ cm)/(cm² sec/Pa)]×10¹³.

23. A donor element for overlaying an image comprising in order a slip layer, an oriented polymer film, and a thermally transferable polymer matrix containing antimicrobial composition.

24. The donor element of claim 23 wherein said thermally transferable polymer comprises a polyethylene-polyvinylalcohol copolymer.

25. The donor element of claim 23 wherein said thermally transferable polymer comprises two or more layers of polymer.

26. The donor element of claim 23 wherein said thermally transferable polymer further comprises thermally expandable polymer beads.

27. The donor element of claim 23 wherein said antimicrobial composition comprises benzoic acid, sorbic acid, nisin, thymol, allicin, peroxide, imazalil, triclosan, benomyl, antimicrobial metal-ion exchange material, metal colloid, anhydride, or organic quaternary ammonium salt.

28. The donor element of claim 23 wherein said antimicrobial composition comprises metal-ion exchange material which is a metal-ion exchange material which has been exchanged or loaded with antimicrobial ions.

29. The donor element of claim 28 wherein said metal ion exchange material is zirconium phosphate, metal hydrogen phosphate, sodium zirconium hydrogen phosphate, zeolite, clay, an ion-exchange resin, an ion exchange polymer, porous alumino-silicate, a layered ion-exchange material, or magnesium silicate.

30. The donor element of claim 23 wherein said oriented polymer film further comprises a polymer layer containing an antimicrobial composition.

31. The donor element of claim 23 wherein said thermally transferable polymer comprises a repeating pattern having a roughness of at least 5 micrometers.

32. The donor element of claim 23 wherein said thermally transferable polymer comprises indicia indicating the presence of said antimicrobial composition.

33. A method for forming an antimicrobial packaging element comprising providing;
a protective donor element for overlaying an image, said donor element comprising in order a slip layer, an oriented polymer film, and a thermally transferable polymer matrix containing antimicrobial composition,
a dye donor element for printing an image, said dye donor element comprising an oriented polymer film and at least one thermal dye transfer dye, and
a packaging substrate comprising a support layer and a thermal dye receiving layer;
thermal dye transfer printing packaging indicia onto said packaging substrate from said dye donor element, and subsequently over printing said packaging indicia with said protective donor element.
